(19) United States
(12) Patent Application Publication
(10) Pub. No.: US 2024/0207438 A1
(43) Pub. Date: Jun. 27, 2024

(54) LIQUID CRYSTAL-BASED MULTIFUNCTIONAL MICRO ROBOT

(71) Applicants: POSTECH Research and Business Development Foundation, Pohang-si (KR); Industry-Academic Cooperation Foundation, Kunsan National University, Gunsan-si (KR)

(72) Inventors: Young-Ki Kim, Pohang-si (KR); Sangmin Jeon, Pohang-si (KR); Chang Yun Son, Pohang-si (KR); Minjae Lee, Gunsan-si (KR); Yena Choi, Pohang-si (KR); Jin-Kang Choi, Pohang-si (KR); Kwang-Suk Oh, Pohang-si (KR); Hyunsoo Han, Pohang-si (KR); Eunsu Cho, Pohang-si (KR)

(21) Appl. No.: 18/376,894

(22) Filed: Oct. 5, 2023

(30) Foreign Application Priority Data
Dec. 23, 2022 (KR) 10-2022-0183795

(51) Int. Cl. A61K 47/69; A61K 41/00; A61K 47/68

(52) U.S. Cl. CPC A61K 47/6901; A61K 41/00; A61K 47/6835; A61K 47/6903

(57) ABSTRACT

Disclosed is a liquid crystal-based multifunctional micro robot including a micro structure including at least one topological defect, an adherent micro particle that is bound to the topological defect and induces self-assembly of a target including a bacterium and a target material based on an antigen-antibody reaction, a physical binding reaction, or a chemical binding reaction, and the bacterium that is attached to the adherent micro particle and provides self-power so that the micro structure approaches the target material.

### CROSS-REFERENCE TO RELATED APPLICATIONS

This application claims priority under 35 U.S.C. § 119 to Korean Patent Application No. 10-2022-0183795 filed on Dec. 23, 2022, in the Korean Intellectual Property Office, the disclosures of which are incorporated by reference herein in their entireties.

### BACKGROUND

Embodiments of the present disclosure described herein relate to a liquid crystal-based multifunctional micro robot.

“The present disclosure is derived from a study conducted as a part of personal basic research of the Ministry of Science and ICT (unique project number: 1711194882, detailed project number: RS-2023-00212739, research project name: development of multifunctional micro robot based on self-assembly of bacteria, host organization: Pohang University of Science & Technology, and research period: 2023.03.01 to 2024.02.29).”

“The present disclosure is derived from a study conducted as a part of personal basic research of the Ministry of Science and ICT (unique project number: 1711188603, detailed project number: 2021R1A2C2095010, research project name: development of high-uniform perovskite nanocrystal mass synthesis method using mesogenic fluid-based microfluidic system, host organization: Pohang University of Science & Technology, and research period: 2023.03.01 to 2024.02.29).”

“The present disclosure is derived from a study conducted as a part of group research support of the Ministry of Science and ICT (unique project number: 1711187151, detailed project number: 2021R1A4A1030944, research project name: research on stable/stretchable/semi-metallic organic semiconductor materials through sequential counterion exchange doping, host organization: Pohang University of Science & Technology, and research period: 2023.03.01 to 2024.02.29).”

Micro robots are next-generation high value-added future technologies and are actively used in various fields, and examples of main application fields thereof include a medical field and a disaster/military exploration field.

In the medical field, a technology of releasing drugs by moving micro robots to locations which surgical instruments are difficult to approach with surgical techniques, a technology of collecting cancer cells, or the like have been developed. In the disaster/military exploration field, a technology of collecting information by moving micro robots through a gap between the ground and a building or a small gap through which the micro robots may enter the building has been developed.

However, technologies of micro robots according to the related art have the following problems.

First, the micro robots according to the related art are manufactured based on electronic devices. In the medical field, as a repulsion to input electronic devices into a body of a patient has increased, the demand for non-electronic device based micro robots has increased.

In addition, in micro robot-related technologies according to the related art, since a micro circuit should be integrated based on an electronic device, a complicated manufacturing process is necessarily accompanied, and external power and fuel such as battery/light/compound are essential for driving. Further, precise driving control is difficult due to the limitation of an integrated part of the micro circuit, and accordingly, it has low functionality.

Accordingly, research on micro robots based on non-electronic devices and capable of overcoming the above limitations have been actively conducted worldwide. However, currently, in non-electronic device based micro robot related technologies, the external power and fuel are still required for driving, and precise control of driving and implementation of multifunctionality are difficult. A liquid crystal-based micro robot according to the present disclosure is based on a non-electronic device, has an easy manufacturing process, may be driven itself without the external power and fuel, and may be implemented to have various functions such as the precise control of driving, and release of drugs, collection, and concentration.

### SUMMARY

Embodiments of the present disclosure provide a liquid crystal-based multifunctional micro robot having improved reliability and functionality in performance and functions required for a non-electronic device-based micro robot and having improved self-position recognition, self-detection technology, self-movement technology, and self-drug release characteristics in a non-structural environment.

According to an embodiment, a liquid crystal-based multifunctional micro robot includes a micro structure including at least one topological defect, an adherent micro particle that is bound to the topological defect and induces self-assembly of a target including a bacterium and a target material based on an antigen-antibody reaction, a physical binding reaction, or a chemical binding reaction, and the bacterium that is attached to the adherent micro particle and provides self-power so that the micro structure approaches the target material.

Further, the micro structure may have a fluid mold core structure made of liquid crystal molecules and may include a carrier stored inside the micro structure.

Further, the micro structure may have a chain core structure including a plurality of mesogenic polymer chains having a ring shape.

Further, the adherent micro particle may be self-assembled to the at least one topological defect disposed on a preset surface area of the micro structure.

Further, the preset surface area may be at least one pole area selected from one side hemisphere pole area and the other side hemisphere pole area of the micro structure.

Further, the adherent micro particle may be a micro particle coated with any one material selected from an antibody specifically bound to surface protein of the target, an aptamer having the same properties as the antibody, an organic molecule specifically bound to the target, and an adsorption structure that adsorbs the target.

Further, the adherent micro particle may include a first adherent micro particle that is bound to the topological defect disposed on the one side hemisphere pole area of the micro structure and provides a first attachment point to which the bacterium is attached and a second adherent micro particle that is bound to the topological defect disposed on the other side hemisphere pole area of the micro structure and provides a second attachment point to which the target material is attached.

Further, at least one micro particle selected from the first adherent micro particle and the second adherent micro particle may further include a magnetic body.

Further, the bacterium may be self-assembled to a distal end of the adherent micro particle based on the antigen-antibody reaction and move the micro structure to an area in which the target material is positioned, through chemotaxis behavior that is a response to a stimulus due to a chemical material concentration difference.

Further, the bacterium may move the micro structure to the area in which the target material is positioned, through aerotaxis behavior that is a response to a stimulus due to an oxygen concentration difference and a response to a stimulus due to an oxygen saturation difference.

Further, the bacterium may move the micro structure to the area in which the target material is positioned, through thermotaxis behavior that is a response to a stimulus due to a temperature gradient.

Further, the bacterium may move the micro structure to the area in which the target material is positioned, through rheotaxis behavior that is a response to a stimulus due to a flow rate gradient.

Further, the bacterium may move the micro structure to the area in which the target material is positioned, through magnetotaxis behavior that is a response to a stimulus due to a magnetic field.

### BRIEF DESCRIPTION OF THE FIGURES

The above and other objects and features of the present disclosure will become apparent by describing in detail embodiments thereof with reference to the accompanying drawings.

FIG. 1 is a view for describing a schematic structure of a liquid crystal-based multifunctional micro robot according to an embodiment of the present disclosure and molecular structures of liquid crystals, additives, and antibodies used in the present disclosure.

FIG. 2 is a view for describing an orientation of internal liquid crystal molecules of micro structures that may be implemented according to an embodiment of the present disclosure and a structure of topological defects according thereto.

FIG. 3 is a view for describing self-assembly of adherent micro particles bound to the topological defects of the micro structures according to an embodiment of the present disclosure in comparison with a general structure.

FIG. 4 is a view for describing a smart detecting and tracking system and a smart release system using the liquid crystal-based multifunctional micro robot according to an embodiment of the present disclosure.

FIG. 5 is a view for describing a go-stop system of the liquid crystal-based multifunctional micro robot according to an embodiment of the present disclosure.

FIG. 6 is a view for describing a smart scavenger system, a smart sterilization system, and a smart recovery system using the liquid crystal-based multifunctional micro robot according to an embodiment of the present disclosure.

FIG. 7 is a view illustrating a schematic structure of a liquid crystal-based multifunctional micro robot according to another embodiment of the present disclosure.

FIG. 8 is a view for describing a smart release system using the liquid crystal-based multifunctional micro robot according to another embodiment of the present disclosure.

FIG. 9 is a view for describing pathogenic bacteria capture system using the liquid crystal-based multifunctional micro robot according to another embodiment of the present disclosure.

FIG. 10 is a view illustrating an experimental result for identifying self-assembly characteristics of bacteria by the adherent micro particles of the liquid crystal-based multifunctional micro robot according to an embodiment of the present disclosure.

FIG. 11 is a view illustrating a binding structure and self-driving of the liquid crystal-based multifunctional micro robot according to an embodiment of the present disclosure.

FIG. 12 is a view illustrating an experimental result for identifying self-tracking characteristics according to self-power of the liquid crystal-based multifunctional micro robot according to an embodiment of the present disclosure and chemotaxis of the bacteria.

FIG. 13 is a view illustrating an experimental result for identifying a speed according to the number of self-assembled bacteria in the liquid crystal-based multifunctional micro robot according to an embodiment of the present disclosure.

### DETAILED DESCRIPTION

Other advantages and features of the present disclosure and a method of achieving the advantages and the features will become apparent with reference to an embodiment described below in detail in conjunction with the accompanying drawings. However, the present disclosure is not limited to an embodiment described below but may be implemented in various forms, an embodiment merely makes the disclosure of the present disclosure complete and is provided to completely inform the scope of the present disclosure to those skilled in the art to which the present disclosure belongs, and the present disclosure is merely defined by the scope of the appended claims.

Even when not defined, all terms (including technical or scientific terms) used herein have the same meanings as those generally accepted by a universal technology in the related art to which the present disclosure pertains. Terms defined by general dictionaries may be interpreted to have the same meanings as those in the related art and/or a text of the present application and will not be conceptualized or excessively formally interpreted even when the expression is not clearly defined herein.

Terms used herein are intended to describe an embodiment and are not intended to limit the present disclosure. In the present specification, a singular form also includes a plural form unless specifically mentioned in a phrase. The term “include” used therein and/or various conjugations of this verb, such as “including” and “included”, do mean that a composition, an ingredient, a component, a step, an operation and/or an element, which is mentioned, does not exclude the presence or addition of one or more other compositions, ingredients, components, steps, operations and/or elements. In the present specification, the term “and/or” refers to each of listed components or various combinations thereof.

Hereinafter, an embodiment of the present disclosure will be described in detail with reference to the accompanying drawings.

In addition, in description of the present disclosure, when it is determined that the detailed description of widely known related configuration or function may make the subject matter of the present disclosure unclear, the detailed description will be omitted.

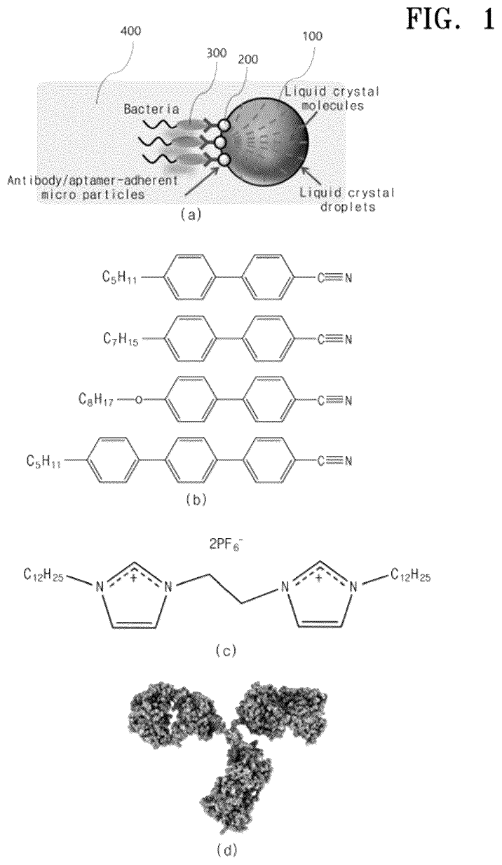

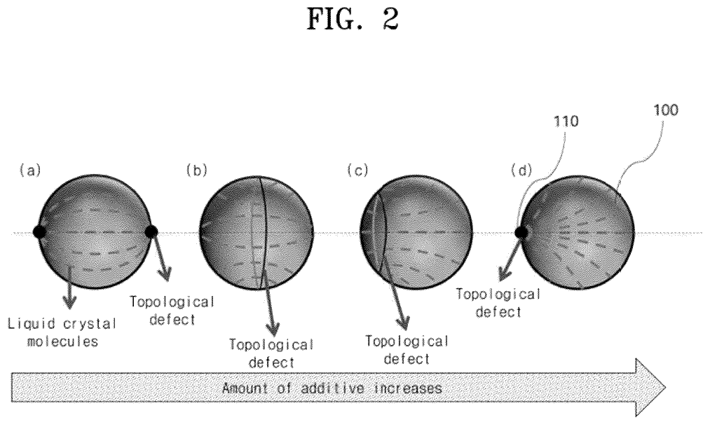

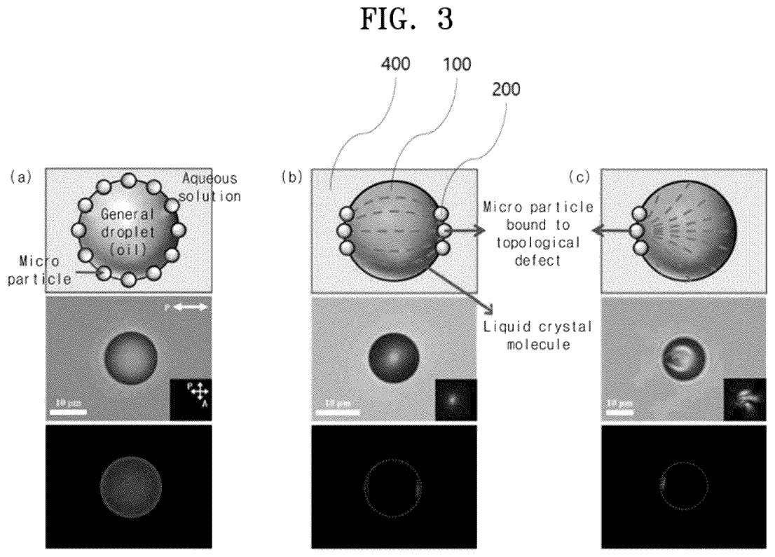

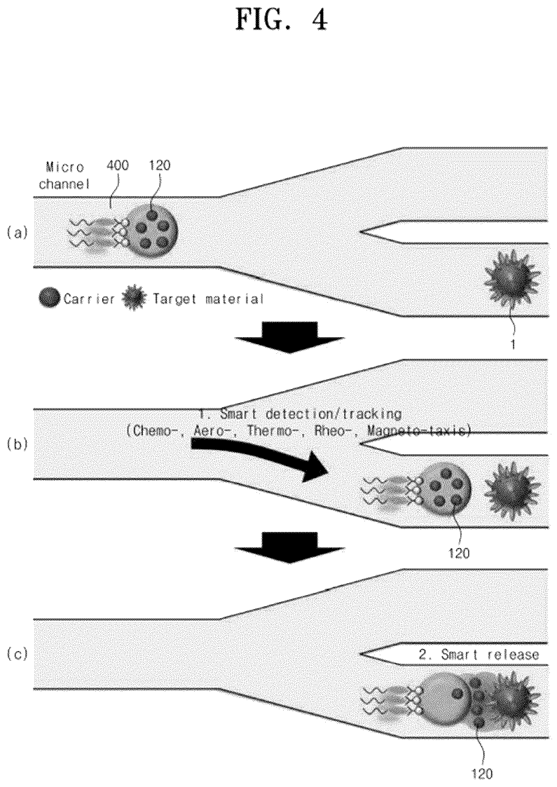

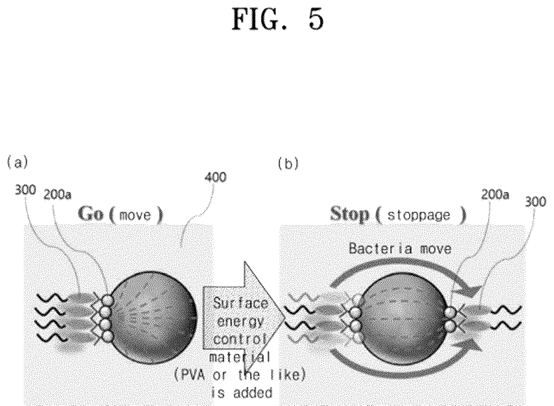

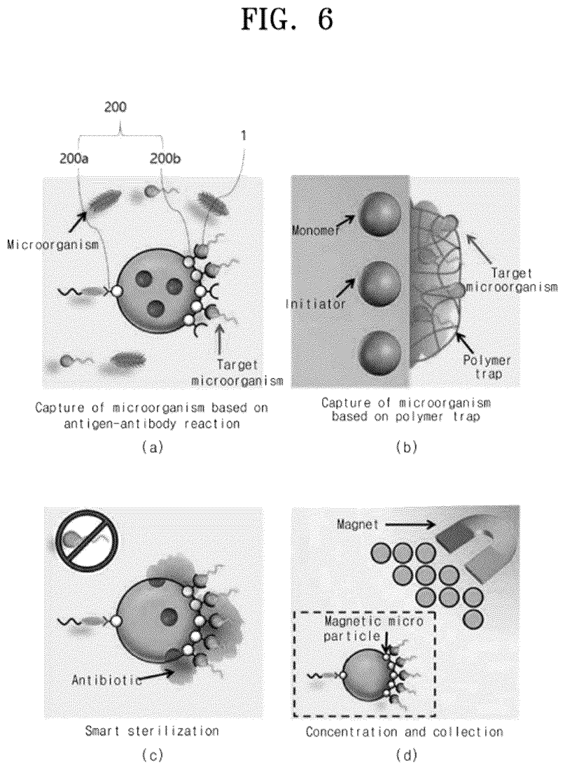

FIG. 1 is a view for describing a schematic structure of a liquid crystal-based multifunctional micro robot according to an embodiment of the present disclosure and molecular structures of liquid crystals, additives, and antibodies used in the present disclosure, FIG. 2 is a view for describing an orientation of internal liquid crystal molecules of micro structures that may be implemented according to an embodiment of the present disclosure and a structure of topological defects according thereto, FIG. 3 is a view for describing self-assembly of adherent micro particles bound to the topological defects of the micro structures according to an embodiment of the present disclosure in comparison with a general structure, FIG. 4 is a view for describing a smart detecting and tracking system and a smart release system using the liquid crystal-based multifunctional micro robot according to an embodiment of the present disclosure, FIG. 5 is a view for describing a go-stop system of the liquid crystal-based multifunctional micro robot according to an embodiment of the present disclosure, and FIG. 6 is a view for describing a smart scavenger system, a smart sterilization system, and a smart recovery system using the liquid crystal-based multifunctional micro robot according to an embodiment of the present disclosure.

FIG. 1A is a view illustrating the schematic structure of the liquid crystal-based multifunctional micro robot according to an embodiment of the present disclosure, FIG. 1B is a view illustrating the molecular structure of the liquid crystal used in the present disclosure, FIG. 1C is a view illustrating the molecular structure of the additive used in the present disclosure, and FIG. 1D is a view illustrating the molecular structure of the antibody used in the present disclosure.

FIGS. 2A to 2D are views illustrating various arrangements of topological defects formed on the surface of the micro structure according to an embodiment of the present disclosure.

First, referring to FIGS. 1 to 6, the liquid crystal-based multifunctional micro robot according to an embodiment of the present disclosure may include a micro structure 100 including at least one topological defect 110, adherent micro particles 200 that are bound to the topological defect 110 and induce self-assembly of targets including bacteria 300 and a target material 1 based on an antigen-antibody reaction, a physical binding reaction or a chemical binding reaction, and the bacteria 300 that are attached to the adherent micro particles 200 and provide self-power so that the micro structure 100 may approach the target material 1.

First, the micro structure 100 may correspond to rounded fluid droplets having a micro size, and in an embodiment of the present disclosure, a liquid crystal may be used as a material of the droplets.

Here, the liquid crystal may be at least one selected from the group consisting of nematic liquid crystals, smectic liquid crystals, cholesteric liquid crystals, and lyotropic liquid crystals, and any liquid crystal may be used without limitation as long as the liquid crystal has a liquid crystal phase within a specific temperature range.

In detail, the micro structure 100 may have a fluid mold core structure made of liquid crystal molecules, and in an embodiment of the present disclosure, the micro structure 100 may be made of E7 liquid crystal molecules having a molecular structure illustrated in FIG. 1B.

Further, as illustrated in FIG. 2, the micro structure 100 may have the at least one topological defect 110, the topological defect 110 may be formed in a selected partial area on the surface of the micro structure 100, and the partial area in which the topological defect 110 is formed may have higher surface energy density than those of the other areas except for the corresponding area.

In detail, at least one topological defect 110 may be disposed on a preset surface area of the micro structure 100 by adjusting surface energy of the micro structure 100, and a form and position of the topological defect 110 may be determined by an orientation of internal liquid crystal molecules of the micro structure 100.

In this case, the orientation of the internal liquid crystal molecules in a stable state may be determined by an orientation of liquid crystal molecules on a structure interface, and the orientation of the liquid crystal molecules on the structure interface may be controlled by adjusting surface energy of the structure interface.

That is, the form and position of topological defect 110 may be determined by adjusting the surface energy of the structure interface, and the surface energy may be adjusted by an additive adsorbed on the structure interface or contained therein. Here, the additive includes materials known to interact with the liquid crystal molecules, such as amphiphiles, organic ionic plastic crystals, lipids, aptamers, antibodies, cholesterol, carbon nanotubes, graphene, and polyvinyl alcohol (PVA).

For example, when the micro structure 100 is formed in an aqueous solution containing no additives, the liquid crystal molecules are oriented in a horizontal direction on a droplet interface, and as illustrated in FIG. 2A, point-shaped topological defects are formed at both poles.

In addition, when the amphiphiles such as organic ionic plastic crystals or sodium dodecyl sulfate (SDS) having the molecular structure illustrated in FIG. 1C are added to the micro structure 100, the orientation of the liquid crystal molecules on the structure interface is inclined from a horizontal direction to a vertical direction. An inclined angle increases according to a concentration of the additive, and when the concentration is greater than or equal to a predetermined concentration, a vertical orientation is achieved.

As illustrated in FIG. 2B, in the inclined orientation of the liquid crystal molecules, a ring-shaped topological defect 110 may be formed in a center of a surface of the structure, and as the concentration of the additive increases, the ring-shaped topological defect 110 moves toward a pole as illustrated in FIG. 2C. When the ring-shaped topological defect 110 reaches the pole, as illustrated in FIG. 2D, the micro structure 100 is stabilized in the form of having the point-shaped topological defect at one pole.

Meanwhile, when the PVA is added as the additive, the micro structure 100 may be stabilized from a form in which the micro structure 100 has the point-shaped topological defect at one pole to a form in which the micro structure 100 has the point-shaped topological defects at both poles.

In other words, the topological defect 110 may be formed in a point shape in at least one of one side hemisphere pole area and the other side hemisphere pole area on a surface of the micro structure 100 or formed in a ring shape in at least one of one side hemisphere area and the other side hemisphere area on the surface of the micro structure 100, and areas in which the topological defects 110 are formed may have relatively high surface energy densities.

In an embodiment of the present disclosure, the preset surface area may be at least one pole area selected from the one side hemisphere pole area and the other side hemisphere pole area of the micro structure 100, and the one side hemisphere pole area and the other side hemisphere pole area are arranged to face each other.

FIG. 3A is a schematic image illustrating micro particles bound onto a surface of a general oil structure, a polarized light micro graph thereof, and a fluorescence micro graph illustrating distribution of the micro particles, and FIGS. 3B and 3C are images of micro particles bound onto a surface of the micro structure according to an embodiment of the present disclosure, polarized light micro graphs thereof, and fluorescence micro graphs illustrating distribution of the micro particles. As illustrated in FIG. 3A, when the oil structure to which the micro particles are bound is dispersed in the aqueous solution, in the case of the general oil structure, the topological defect 110 cannot be controlled, and thus the micro particles are uniformly distributed over the entire area of the surface of the oil structure.

On the other hand, when the micro structure 100 according to an embodiment of the present disclosure is used as a body of a micro robot, the topological defects 110 may be controlled to be arranged on a preset surface area. Accordingly, micro particles such as the adherent micro particles 200 are bound to the topological defect 110, and thus the micro particles may be controlled to be arranged on a desired surface area.

In detail, as illustrated in FIG. 3B, the topological defects 110 in the micro structure 100 may be controlled to face each other in one side hemisphere pole area and the other side hemisphere pole area of the droplets. Further, as illustrated in FIG. 3C, the topological defects 110 in the micro structure 100 may be controlled to be disposed only in the one side hemisphere pole area of the droplets. Accordingly, the adherent micro particles 200 may be bound to the one side hemisphere pole area and the other side hemisphere pole area of the droplets or bound only to the one side hemisphere pole area of the droplets.

Meanwhile, the micro structure 100 according to an embodiment of the present disclosure may further include a carrier 120 stored inside the micro structure 100. Here, the carrier 120 may be a liquid or solid carrier.

The carrier 120 may be one material selected from a drug encapsulation bead that encapsulates a drug, a therapeutic bacterium encapsulation bead that encapsulates therapeutic bacteria, and a monomer bead that may capture the target material 1 by causing polymerization or gelation. Any material may be used as the carrier 120 without limitation as long as the material may react with the target material 1 without being mixed with the liquid crystals.

The drug may include an anticancer drug, and specifically, may include anthracycline, doxorubicin, daunorubicin, idarubicin, and actinomycin D.

In addition, the drug may include an antibiotic drug, and any therapeutic pharmaceutical composition may be used as the drug without limitation.

The monomer bead may include a polymer monomer, an initiator, and a crosslinking agent. Specifically, the polymer monomer may include N-isopropylacrylamide (NIPAm), sodium alginate, and chitosan, and any material may be used as the polymer monomer as long as the material may cause polymerization or gelation.

Next, the adherent micro particles 200 according to an embodiment of the present disclosure may be micro particles coated with one material selected from antibodies specifically bound to surface proteins of the targets, aptamers having the same properties as those of the antibodies, organic molecules specifically bound to the target, and adsorption structures that adsorb the targets.

In detail, the adherent micro particles 200 may include one selected from the micro particles, the antibodies, the aptamers, the organic molecules, and the adsorption structures. The micro particles may be particles having a micro or nano size and may be used without limitation as long as the micro particles may be coated with the antibodies, the aptamers, the organic molecules, or the adsorption structures without being mixed with the liquid crystals.

Here, the organic molecules may be made of a material that is specifically bound to a heavy metal, the adsorption structures may be made of a material that adsorbs acidic substances, basic substances and radioactive substances, and the aptamers may be a single-stranded oligonucleic acid.

In an embodiment of the present disclosure, the antibody may include a bacteria-derived antibody having a molecular structure illustrated in FIG. 1D, and the bacteria-derived antibody may be Goat anti-Salmonella CSA-1.

Further, the micro particles may include any one material selected from silica, iron oxide, polystyrene, perovskite, and glass.

In addition, the adherent micro particles 200 may be formed by coating the micro particles with the antibodies or aptamers through stirring of a mixture of the antibodies or the aptamers and the micro particles based on 1-ethyl-3-(3-dimethylaminopropyl)carbodiimide / N-hydroxysuccinimide(EDC/NHS) reaction.

In an embodiment of the present disclosure, the adherent micro particles 200 may further include a magnetic body for collecting captured micro organisms.

Meanwhile, in an embodiment of the present disclosure, the target may be the bacteria 300 that provide power to the micro structure 100, and furthermore, may further include the target material 1 to be captured, sterilized, concentrated, and collected.

In this case, the bacteria 300 are mobile bacteria and are distinguished from pathogenic bacteria and therapeutic bacteria.

The target material 1 may include pathogenic viruses, germs, fungi, and protozoa or may be microorganisms including human reproductive cells such as sperm or eggs and cancer cells exhibiting abnormal growth, and examples of the target material 1 are not limited as long as the target material 1 reacts with the adherent micro particles 200 through the antigen-antibody reaction, the physical binding reaction, or the chemical binding reaction.

Further, the target material 1 may also include bacteria having other characteristics except for bacteria having mobile characteristics to provide self-power to micro robots, and may specifically include pathogenic bacteria.

Further, the target material 1 may include heavy metals, acidic substances, basic substances, and radioactive substances that cause environmental pollution.

In addition, the adherent micro particles 200 are self-assembled with at least one topological defect 110 disposed on a predetermined surface area of the micro structure 100.

As an example, when the topological defect 110 is formed in the one side hemisphere pole area and the other side hemisphere pole area of the micro structure 100, the adherent micro particles 200 are bound to the formed topological defect 110 to provide an attachment point to which the bacteria 300 and the target material 1 may be attached.

In detail, as illustrated in FIG. 6, the adherent micro particles 200 provide a first attachment point which is bound to the topological defect 110 disposed on the one side hemisphere pole area of the micro structure 100 and to which the bacteria 300 may be attached and a second attachment point which is bound to the topological defect 110 disposed on the other side hemisphere pole area of the micro structure 100 and to which the target material 1 may be attached.

That is, the adherent micro particles 200 may include first adherent micro particles 200a that provide the first attachment point and second adherent micro particles 200b that provide the second attachment point. The first adherent micro particles 200a may be micro particles coated with any one material selected from antibodies that are specifically bound to surface proteins of the bacteria 300 and aptamers having the same properties as those of the antibodies. The second adherent micro particles 200b may be micro particles coated with any one material selected from antibodies that are specifically bound to surface proteins of the target material 1, aptamers having the same properties as those of the antibodies, organic molecules that are specifically bound to the target material 1, and adsorption structures that adsorb the target material 1.

In an embodiment of the present disclosure, the first adherent micro particles 200a that provide the first attachment point may be micro particles coated with Goat anti-Salmonella CSA-1 that is the bacteria-derived antibody.

In addition, at least one selected from the first adherent micro particles 200a and the second adherent micro particles 200b may further include the magnetic body.

As another example, when the topological defect 110 is formed in the one side hemisphere pole area and the other side hemisphere pole area of the micro structure 100, the adherent micro particles 200 may be bound to the formed topological defect 110 to provide the attachment point to which only the bacteria 300 may be attached.

In detail, as illustrated in FIG. 5, the adherent micro particles 200 may be bound to the topological defect 110 disposed in at least one selected from the one side hemisphere pole area and the other hemisphere pole area of the micro structure 100 and thus provide the first attachment point to which the bacteria 300 may be attached.

In this case, the adherent micro particles 200 may include the first adherent micro particles 200a that provide the first attachment point, and the first adherent micro particles 200a are micro particles coated with any one selected from the antibodies that are specifically bound to the surface proteins of the bacteria 300 and the aptamers having the same properties as those of the antibodies.

Next, the bacteria 300 according to an embodiment of the present disclosure are attached to the adherent micro particles 200 to provide self-power together with directionality to the micro structure 100.

The bacteria 300 may be self-assembled at distal ends of the adherent micro particles 200 based on the antigen-antibody reaction.

The bacteria 300 are attached to the adherent micro particles 200 to form one bacterial motor, may exhibit random behavior in normal times, and may set a target and move by any one of stimuli including a chemical material concentration difference, an oxygen concentration difference, an oxygen saturation difference, a temperature gradient, a flow rate gradient, and a magnetic field.

In detail, the bacteria 300 may be attached to the first adherent micro particles 200a and may move the micro structure 100 to an area in which the target material 1 is positioned, through chemotaxis behavior that is a response to a stimulus due to the chemical material concentration difference.

In addition, the bacteria 300 may move the micro structure 100 to the area in which the target material 1 is positioned, through aerotaxis behavior that is a response to a stimulus due to the oxygen concentration difference and a response to a stimulus due to the oxygen saturation difference.

Further, the bacteria 300 may move the micro structure 100 to the area in which the target material 1 is positioned, through thermotaxis behavior that is a response to a stimulus due to an ambient temperature difference and may move the micro structure 100 to the area in which the target material 1 is positioned, through rheotaxis behavior that is a response to a stimulus due to the flow rate gradient.

In addition, the bacteria 300 may move the micro structure 100 to the area in which the target material 1 is positioned, through magnetotaxis behavior that is a response to a stimulus due to a magnetic field.

Accordingly, the liquid crystal-based multifunctional micro robot according to an embodiment of the present disclosure may flow inside an accommodation space 400 to target the target material 1 by flagellum movement of the bacteria 300 attached to the adherent micro particles 200 bound to the micro structure 100.

### <Smart Detecting and Tracking System and Smart Release System>

FIGS. 4A and 4B are views for describing a smart detecting and tracking system of the liquid crystal-based multifunctional micro robot, and FIG. 4C is a view for describing a smart release system of the liquid crystal-based multifunctional micro robot.

Referring to FIG. 4, the liquid crystal-based multifunctional micro robot according to an embodiment of the present disclosure is formed by attaching the bacteria 300 serving as motors to the micro structure 100-based body. As the bacteria 300 have chemotaxis, aerotaxis, rheotaxis, and magnetotaxis, which are properties of moving toward the target, the micro robot may detect and track the target material 1, which is a specific target, due to characteristics of the bacteria 300 even without any electronic devices.

In detail, as illustrated in FIGS. 4A to 4C, the liquid crystal-based multifunctional micro robot according to an embodiment of the present disclosure is formed such that the first adherent micro particles 200a are bound to the one side hemisphere pole area of the micro structure 100. The bacteria 300 are attached to the one side hemisphere pole area, and the carrier 120 is included therein.

The micro robot may have a multifunctional feature capable of detecting and tracking the target material 1, approaching the target material 1, and releasing the carrier 120 stored inside the micro structure 100.

Accordingly, the liquid crystal-based multifunctional micro robot according to an embodiment of the present disclosure may be utilized for the smart detecting and tracking system that self-detects and then self-tracks the target material 1 using self-power through the chemotaxis behavior, the aerotaxis behavior, the thermotaxis behavior, the rheotaxis behavior, and the magnetotaxis behavior of the bacteria 300 and may be utilized for the smart release system that releases the carrier 120 to the vicinity of the target using a carrier storing and releasing function of the micro structure 100.

### <Go-Stop System>

FIG. 5A is a view for describing movement of the liquid crystal-based multifunctional micro robot, and FIG. 5B is a view for describing stop of the liquid crystal-based multifunctional micro robot.

Referring to FIG. 5, the liquid crystal-based multifunctional micro robot according to an embodiment of the present disclosure may control the behavior by controlling a position of the topological defect 110 of the micro structure 100 by adjusting the surface energy of the micro structure 100.

In detail, as illustrated in FIG. 5A, when the topological defect 110 is disposed only in the one side hemisphere pole area of the micro structure 100, the first adherent micro particles 200a are bound to the one side hemisphere pole area of the micro structure 100, the bacteria 300 are attached to the one side hemisphere pole area, and thus the liquid crystal-based multifunctional micro robot may move.

In addition, as illustrated in FIG. 5B, when the amount of additive adsorbed onto the structure interface is adjusted, that is, when the additive such as the PVA is added to the accommodation space 400, the topological defect 110 may be disposed to face the one side hemisphere pole area and the other side hemisphere pole area of the droplets. In this case, the first adherent micro particles 200a are bound to the one side hemisphere pole area and the other side hemisphere pole area of the micro structure 100, and thus the bacteria 300 may be attached to the one side hemisphere pole area and the other side hemisphere pole area. Accordingly, the bacteria 300 exist in the pole areas on both sides, and thus the movement of the liquid crystal-based multifunctional micro robot may be stopped.

### <Smart Scavenger System, Smart Sterilization System, and Smart Recovery System>

FIGS. 6A and 6B are views illustrating capture of microorganisms using the liquid crystal-based multifunctional micro robot, FIG. 6C is a view illustrating smart sterilization using the liquid crystal-based multifunctional micro robot, and FIG. 6D is a view illustrating concentration and collection of the microorganisms using the liquid crystal-based multifunctional micro robot.

Referring to FIG. 6, the liquid crystal-based multifunctional micro robot according to an embodiment of the present disclosure may be formed such that the bacteria 300 serving as motors are attached to the micro structure 100-based body, and at the same time, the target material 1, which is a target other than the bacteria 300, is attached to the micro structure 100-based body.

In the liquid crystal-based multifunctional micro robot according to an embodiment of the present disclosure, the first adherent micro particles 200a are bound to the one side hemisphere pole area of the micro structure 100, and the second adherent micro particles 200b are bound to the other side hemisphere pole area thereof. The bacteria 300 may be attached to the one side hemisphere pole area, and the target material 1 may be attached to the other side hemisphere pole area.

As illustrated in FIGS. 6A and 6C, in the liquid crystal-based multifunctional micro robot according to an embodiment of the present disclosure, the bacteria 300 are attached to the first adherent micro particles 200a bound to the one side hemisphere pole area of the micro structure 100, and thus a specific target material 1 may be detected and tracked due to the characteristics of the bacteria 300. Further, the target material 1 may be attached to the second adherent micro particles 200b bound to the other side hemisphere pole area of the micro structure 100, and in a state in which the target material 1 is attached thereto, the liquid crystal-based multifunctional micro robot may be utilized for the smart scavenger system and the smart sterilization system that capture and sterilize the microorganisms using the carrier storing and releasing function of the micro structure 100.

In addition, the target material 1 of the liquid crystal-based multifunctional micro robot according to an embodiment of the present disclosure illustrated in FIGS. 6A and 6C may be changed according to the type of the second adherent micro particles 200b bound to the other side hemisphere pole area.

Further, as illustrated in FIG. 6B, when the carrier 120 stored inside the micro structure 100 includes the monomer bead that causes polymerization or gelation, the liquid crystal-based multifunctional micro robot according to an embodiment of the present disclosure forms a polymer trap using the release function of the micro structure 100 without the second adherent micro particles 200b bound to the other side hemisphere pole area of the micro structure 100, and thus may be utilized for the smart scavenger system.

In addition, as illustrated in FIG. 6D, in the liquid crystal-based multifunctional micro robot according to an embodiment of the present disclosure, the second adherent micro particles 200b further include the magnetic body. Thus, the liquid crystal-based multifunctional micro robot may be utilized for the smart recovery system that captures the target material 1 and then be concentrated and collected through magnetism.

That is, the liquid crystal-based multifunctional micro robot according to an embodiment of the present disclosure may capture the target material 1 using the second adherent micro particles 200b bound to the other side hemisphere pole area of the micro structure 100, form the polymer trap by using the carrier releasing function of the micro structure 100 near the target material 1, release and sterilize the carrier 120 near the target material 1 using the carrier releasing function of the micro structure 100 in a state in which the target material 1 is captured, and recover the micro structure 100 through magnetism to concentrate and collect the target material 1, in a state in which the target material 1 is captured.

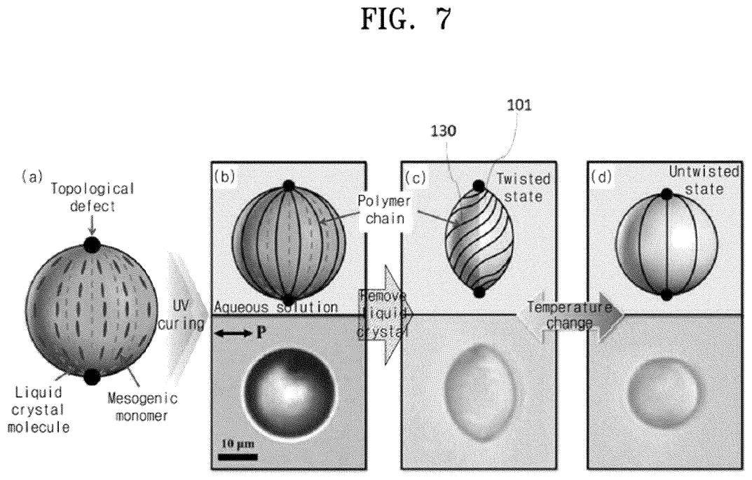

Hereinafter, a schematic structure of a liquid crystal-based multifunctional micro robot according to another embodiment of the present disclosure will be described with reference to FIGS. 7 to 9. However, since the embodiment of FIGS. 7 to 9 is different from the embodiment of the micro structure 100 in that the micro structure includes a polymer chain, a difference therebetween will be mainly described below.

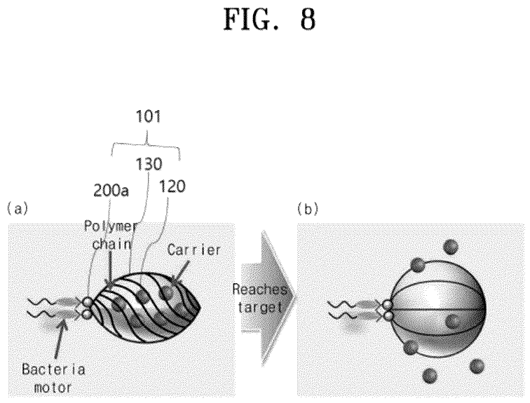

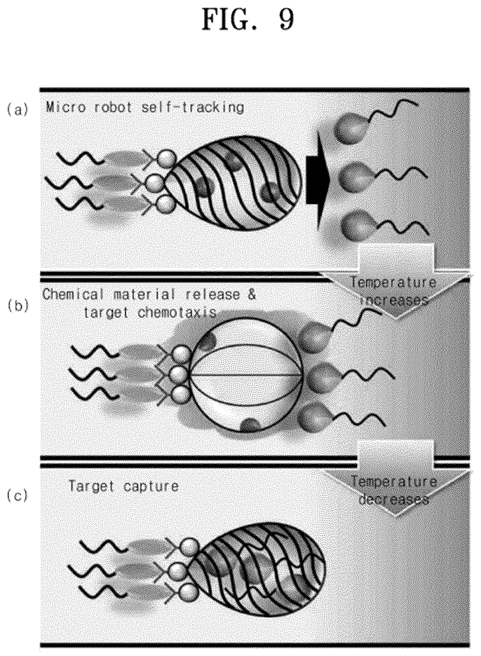

FIG. 7 is a view illustrating a schematic structure of a liquid crystal-based multifunctional micro robot according to another embodiment of the present disclosure, FIG. 8 is a view for describing a smart release system using the liquid crystal-based multifunctional micro robot according to another embodiment of the present disclosure, and FIG. 9 is a view for describing a pathogenic bacteria capture system using the liquid crystal-based multifunctional micro robot according to another embodiment of the present disclosure.

FIG. 7A is a view for describing a polymer monomer included in the micro structure, FIG. 7B is a view for describing the polymer chain included in the micro structure, FIG. 7C is a view for describing a form of the micro structure after liquid crystals are removed at the room temperature, and FIG. 7D is a view for describing a form of the micro structure according to a temperature change.

FIG. 8A is a view for describing the liquid crystal-based multifunctional micro robot before the carrier is released, and FIG. 8B is a view for describing release of the carrier by the liquid crystal-based multifunctional micro robot after the liquid crystal-based multifunctional micro robot reaches a target position.

Referring to FIGS. 7 and 8, in the liquid crystal-based multifunctional micro robot according to another embodiment of the present disclosure, a micro structure 101 may have a chain core structure made of a plurality of mesogenic polymer chains 130 having a ring shape.

In detail, the micro structure 101 may include the mesogenic polymer chains 130 having any one ring shape of a twisted ring shape and a linear ring shape according to the temperature change.

In addition, the micro structure 101 may further include photothermal particles exhibiting a photothermal effect, and the photothermal particles may be any one selected from titanium nitride (TiN) particles, gold nano-particles, and gallium-indium eutectic alloy (EGaIn) particles.

Here, the gallium-indium eutectic alloy is a metallic material that is maintained in a liquid phase even at the room temperature due to a low melting point thereof.

As illustrated in FIGS. 7A and 7B, the micro structure 101 according to another embodiment of the present disclosure may be formed through polymerization of the polymer monomer using liquid crystal droplets as templates, and in an embodiment of the present disclosure, the polymer monomer may be a mesogenic monomer.

In detail, during UV curing, the mesogenic polymer chains 130 derived from the polymer monomer are formed in the liquid crystal droplets according to the orientation of the liquid crystal molecules. Thereafter, when all the liquid crystal molecules used as the templates are removed from the liquid crystal droplets in which the mesogenic polymer chains 130 are formed, the micro structure 101 having improved stability against environmental factors including a temperature, a pH, and fluid flow while preserving orientation characteristics may be obtained.

In addition, the obtained micro structure 101 is formed using, as the templates, the liquid crystal droplets including at least one topological defect 110 and thus may include the topological defect 110 of the removed liquid crystal droplets without change. That is, even when the liquid crystal droplets are removed, the topological defect 110 may remain in the chain core structure.

Referring to FIG. 7C, the obtained micro structure 101 has the chain core structure made of the mesogenic polymer chains 130 having a twisted ring shape at the room temperature, and thus may have elasticity, a high polymer chain density, and an ellipsoidal shape.

In addition, as illustrated in FIG. 7D, a shape of the micro structure 101 according to another embodiment of the present disclosure may be changed so that the micro structure 101 has a low polymer chain density and a spherical shape according to the temperature change, and the shape change may occur at a temperature that satisfies a range of 100° C. to 120° C.

In detail, in the micro structure 101 according to another embodiment of the present disclosure, the polymer chain density of the micro structure 101 at the room temperature may be higher than the polymer chain density of the micro structure 101 at the temperature that satisfies the range of 100° C. to 120° C. That is, a separation distance between the plurality of polymer chains may be changed according to the temperature change, and as the temperature increases, the separation distance between the plurality of polymer chains may increase.

### <Smart Release System in Harsh Environment>

Referring to FIG. 8, the micro structure 101 according to another embodiment of the present disclosure may be used in a smart release system in a harsh environment.

As illustrated in FIG. 8A, the multifunctional micro robot according to another embodiment of the present disclosure is formed by attaching the bacteria 300 serving as the motors to the micro structure 101-based body. As the bacteria 300 have chemotaxis, aerotaxis, thermotaxis, rheotaxis, and magnetotaxis, which are properties of moving toward the target, the micro robot may detect and track the target material 1, which is a specific target, due to the characteristics of the bacteria 300 even without any electronic devices.

In addition, the micro structure 101 may further include the carrier 120 stored therein, and the plurality of mesogenic polymer chains 130 included in the micro structure 101 have a twisted ring shape at the room temperature and may be arranged with a separation distance between the polymer chains through which the carrier 120 may not pass.

In other words, as the micro structure 101 has a high polymer chain density at the room temperature, the carrier 120 may stably move to the vicinity of the target material 1 even in a harsh environment.

Meanwhile, as illustrated in FIG. 8B, at a temperature that satisfies a range of 100° C. to 120° C., the plurality of mesogenic polymer chains 130 included in the micro structure 101 may have a linear ring shape and may be arranged with a separation distance between the polymer chains through which the carrier 120 may pass.

That is, the micro structure 101 may be changed into a spherical shape having a low polymer chain density due to a decrease in elasticity of the plurality of mesogenic polymer chains 130 as the temperature increases.

In detail, the temperature of the micro structure 101 may increase through a photothermal effect that is an effect of absorbing a light beam and emitting heat, and a separation distance between the polymer chains may increase through heat emitted by the photothermal particles by irradiating a light beam having a specific wavelength.

Accordingly, the liquid crystal-based multifunctional micro robot according to another embodiment of the present disclosure may have excellent stability even in harsh environments such as acidic/basic conditions, high temperatures, and high flow rates by utilizing the micro structure 101 as a body of the robot. Here, the harsh environment includes strong acidic and strong basic environment of a pH range of 1 to 14, high temperature environment of 200° ° C. or higher, and high flow rate environment of 100 cm/s.

### <Tracking, Releasing, and Capture System in Harsh Environment>

FIG. 9A is a view for describing a self-tracking system of the liquid crystal-based multifunctional micro robot according to another embodiment, FIG. 9B is a view for describing a releasing system of the liquid crystal-based multifunctional micro robot according to another embodiment, and FIG. 9C is a view for describing a capture system of the liquid crystal-based multifunctional micro robot according to another embodiment.

As illustrated in FIG. 9A, the multifunctional micro robot according to another embodiment of the present disclosure may detect and track the target material 1 that is a specific target due to the characteristics of the bacteria 300, may stably store the carrier 120 as the micro structure 101 has a high polymer chain density, and may be stably driven even in a harsh environment.

As illustrated in FIG. 9B, in the multifunctional micro robot according to another embodiment of the present disclosure, the shape of the micro structure 101 is changed so that the micro structure 101 has a low polymer chain density and a spherical shape due to the temperature increase, and thus the carrier 120 is emitted to the outside. Further, the target material 1 may be introduced into the micro structure 101 through the change in the shape of the micro structure 101 due to the temperature increase. Thereafter, referring to FIG. 9C, the shape of the micro structure 101 is changed so that the micro structure 101 has a high polymer chain density and an elliptical shape due to a temperature decrease, and thus the target materials 1 may be captured inside the droplets.

Materials utilized in the present disclosure may include materials having excellent biometric/environmental suitability. Accordingly, influence of the present disclosure on the environment may be minimized.

Hereinafter, the present disclosure will be described in more detail by accompanying examples. However, the following examples are merely intended to describe the present disclosure, and the scope of the present disclosure is not limited thereby.

### Example 1: Manufacturing of Liquid Crystal-Based Multifunctional Micro Robot 1

First, a liquid crystal mixture is formed by adding about 0.3 wt % of antibody-adherent micro particles, which are the first adherent micro particles, to a mixture of the E7 liquid crystal having a molecular structure illustrated in FIG. 1B and 1 mM organic ionic material, which is an additive having a molecular structure illustrated in FIG. 1C to adjust surface energy of the E7 liquid crystal.

Here, the antibody-adherent micro particles include, as an antibody, Goat anti-Salmonella CSA-1 having the molecular structure illustrated in FIG. 1D. To coat the micro particles with the antibodies, 1 mg of the micro particles, 2 mg of 1-ethyl-3-(3-(dimethylamino)propyl)carbodiimide(EDC), 10 mg of N-hydroxysuccinimide(NHS), and 10 mL of distilled water are mixed and then stirred for two hours. Thereafter, 6 mg of the Goat anti-Salmonella CSA-1 is added to the stirred object, a mixture thereof is stirred for two hours, and thus the antibody-adherent micro particles are prepared.

The liquid crystal mixture is dispersed in the accommodation space through an ultrasonic mixer to form a micro structure having a diameter of 5 μm. In this case, the micro structure may also be formed through a mixer or a microfluidic system.

The arrangement of the topological defect in the formed micro structure is determined depending on the concentration of the additive, the antibody-adherent micro particles, which are the first adherent micro particles that provide the first attachment point to which the bacteria may be attached, are self-assembled and attached to the topological defect disposed in the one side hemisphere pole area of the micro structure, and thus a body of the micro robot is completed.

Thereafter, when the bacteria that are targets corresponding to the antibody-adherent micro particles are added to the accommodation space, the bacteria are self-assembled and thus attached to the antibody-adherent micro particles through the antigen-antibody reaction, and thus a bacteria self-assembled-based micro robot is completed (example 1).

### Example 2: Manufacturing of Liquid Crystal-Based Multifunctional Micro Robot 2

A micro robot is manufactured in the same manner as that of example 1 except that the number of self-assembled bacteria increases by increasing the number of antibody-adherent micro particles in the body of the micro robot in example 1 (example 2).

### Example 3: Manufacturing of Liquid Crystal-Based Multifunctional Micro Robot 3

To form the micro robot for the smart release system, in a process of forming the liquid crystal mixture in example 1, an aqueous carrier solution that does not dissolve in the E7 liquid crystal is added together, and then the micro robot is manufactured in the same manner as that of example 1 (example 3). The size of the generated micro structure and the size of carrier droplets stored therein may be adjusted during the process of forming the liquid crystal mixture in example 1. Further, the topological defect is generated around an inner carrier of the liquid crystal, an elastic repulsive force of the liquid crystal is formed between the carriers, and thus merging of the inner carrier is prevented.

### Example 4: Manufacturing of Liquid Crystal-Based Multifunctional Micro Robot 4

The micro robot is manufactured in the same manner as that of example 1 except that a concentration of the organic ionic material is reduced, germ adherent micro particles that are the second adherent micro particles that provide the second attachment point to which germs other than the bacteria may be attached are further added, and the germs are self-assembled and thus attached to the germ adherent micro particles through the antigen-antibody reaction by adding the germs that are targets corresponding to the germ adherent micro particles to the accommodation space in a process of forming the liquid crystal mixture of example 1 to form a micro robot for the smart scavenger system.

### Example 5: Manufacturing of Liquid Crystal-Based Multifunctional Micro Robot 5

To form a micro robot for the smart release system in a harsh environment, the E7 liquid crystals having the molecular structure illustrated in FIG. 1B, mesogenic monomers (Reactive mesogen 82), mesogenic monomer oligomers (RM82-1,3 PDT), photoinitiators (Irgacure651), and the antibody-adherent micro particles are mixed and dispersed in the aqueous solution. In this case, the concentration of the liquid crystal is 80 wt %, and the mesogenic monomer and the mesogenic monomer oligomer are mixed in a ratio of 1:1 at a concentration of 20 wt %. The photoinitiator is added by 2 wt % of the mesogenic monomer. Here, the antibody-adherent micro particles are coated through the same process as that of example 1 and are mixed by 0.3 wt %.

The aqueous solution is mixed with 5 wt % of PVA to adjust surface energy of the liquid crystal.

The dispersed liquid crystal mixture is polymerized under 365 nm UV for two hours. Thereafter, after the liquid crystal mixture is precipitated, a supernatant is removed. Thereafter, the E7 liquid crystal is removed by adding ethanol.

Thereafter, when the bacteria that are the targets corresponding to the antibody-adherent micro particles are added to the accommodation space, the bacteria are self-assembled and thus attached to the antibody-adherent micro particles through the antigen-antibody reaction, and thus the liquid crystal-based multifunctional micro robot is completed (example 5).

### Comparative Example 1: Manufacturing of Liquid Crystal-based Micro Robot 1

A micro robot is manufactured in the same manner as that of example 1 except for an operation of attaching the bacteria to the body of the micro robot in example 1 through the self-assembly (comparative example 1).

### Comparative Example 2: Manufacturing of Liquid Crystal-based Micro Robot 2

A micro robot is manufactured in the same manner as that of example 1 except that the micro structure having a diameter of 10 μm is used as the body of the micro robot, and the number of self-assembled bacteria increases by increasing the number of antibody adherent micro particles in example 1 (comparative example 2).

### <Experimental Example 1> Analysis by Optical Microscope

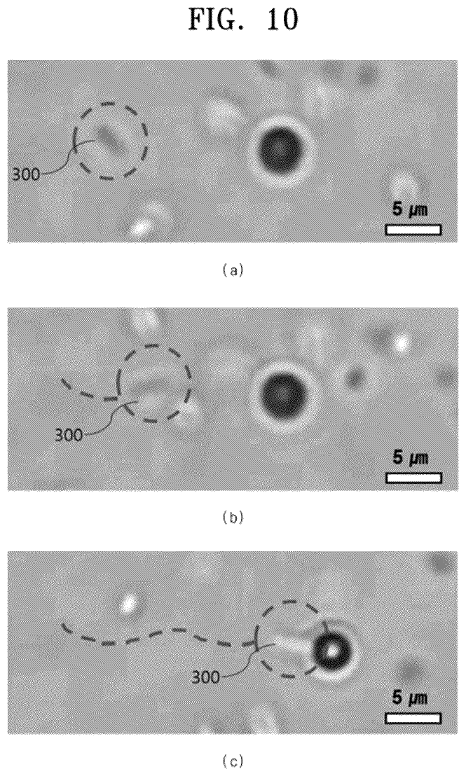

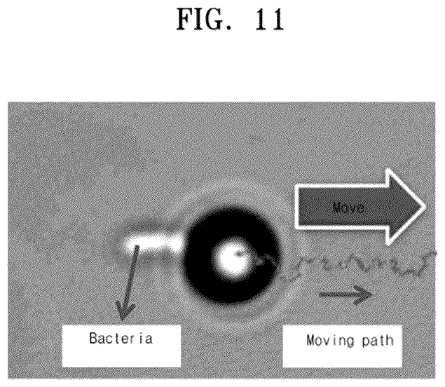

To identify antigen-antibody-based self-assembly characteristics of the liquid crystal-based multifunctional micro robot according to an embodiment of the present disclosure, a binding process between the body (a first adherent micro particle/micro structure complex) of the micro robot formed through example 1 and the bacteria as the targets was photographed in real time using an optical microscope, and a result thereof was illustrated in FIGS. 10 and 11.

FIG. 10 is a view illustrating an experimental result for identifying self-assembly characteristics of bacteria by the adherent micro particles of the liquid crystal-based multifunctional micro robot according to an embodiment of the present disclosure, and FIG. 11 is a view illustrating a binding structure and self-driving of the liquid crystal-based multifunctional micro robot according to an embodiment of the present disclosure.

FIGS. 10A, 10B, and 10C are views for describing a process in which the target bacteria are self-assembled to the first adherent micro particles.

First, referring to FIG. 10, it was identified that the target bacteria 300 exhibiting random behavior in the aqueous solution were self-assembled to the first adherent micro particle/micro structure complex based on the antigen-antibody reaction.

Next, as illustrated in FIG. 11, it was identified that the bacteria 300 as the targets were not attached to the entire surface of the micro structure but were attached to only the first attachment point provided by the first adherent micro particles bound to the micro structure, and therefore, the micro structure moved in one direction.

### <Experimental Example 2> Analysis of Self-Behavior and Chemotaxis Behavior Through Bacteria

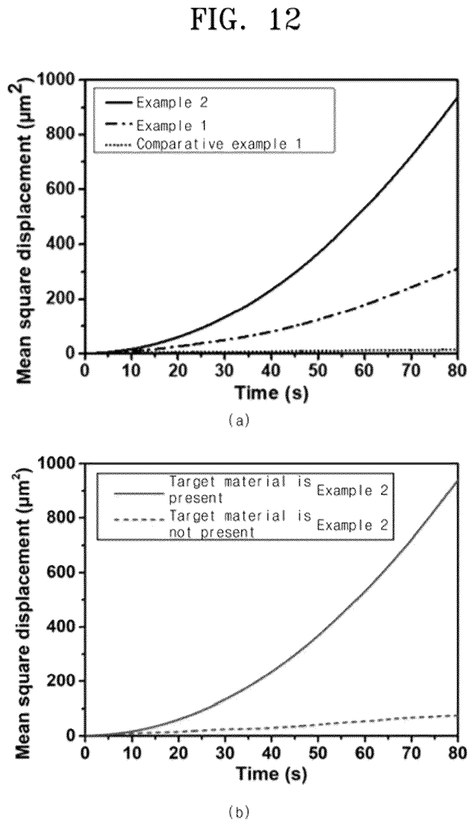

To identify self-behavior and chemotaxis behavior characteristics of the liquid crystal-based multifunctional micro robot according to an embodiment of the present disclosure, the behavior of the micro robot (the first adherent micro particle/micro structure complex to which the bacteria are self-assembled) manufactured through example 1, example 2, and comparative example 1 was photographed in real time using the optical microscope, and as the corresponding image is pre-processed, a trajectory and a speed field of the micro robot were acquired. In detail, the behavior of the micro robot was analyzed based on mean square displacement (MSD) calculated using the trajectory of the micro structure. Further, a MSD-time lag graph for the trajectory of the micro robot was acquired and then was illustrated in FIG. 12. Here, the number of bacteria self-assembled to the micro structure of example 2 is two.

In more detail, in the self-behavior analysis, the behavior of the micro robot according to example 1, example 2, and comparative example 1 in a state in which the target material is provided was analyzed, and in the chemotaxis behavior, the behavior of the micro robot according to example 2 in a state in which the target material is provided and a state in which the target material is not provided was analyzed.

FIG. 12 is a view illustrating an experimental result for identifying self-tracking characteristics according to self-power of the liquid crystal-based multifunctional micro robot according to an embodiment of the present disclosure and chemotaxis of the bacteria.

FIG. 12A is a result obtained by analyzing the behavior of the micro robot according to example 1, example 2, and comparative example 1, and FIG. 12B is a result obtained by analyzing the behavior according to the presence or absence of the target material in example 2.

Referring to FIG. 12A, it was identified that the micro robot of example 1 has a structure in which the bacteria are attached to the micro structure, and a graph increases in the form of an exponential function and illustrates diffusion behavior in one direction. On the other hand, it was identified that the micro robot according to comparative example 1 is the micro robot to which the bacteria are not attached and which exhibits a general Brownian motion.

Further, one or more bacteria might be self-assembled to the first adherent micro particle/micro structure complex, and it was identified that, as the number of self-assembled bacteria increases, the diffusion behavior of the micro robot of example 2 in one direction is further improved.

In addition, referring to FIG. 12B, it was identified that, when the chemotactic bacteria are self-assembled to the micro structure, the diffusion behavior of the micro robot of example 2 corresponds to a direction in which a chemical material is positioned. This means that the behavior of the micro robot is caused by the chemotactic behavior of bacteria.

In addition, it was identified that, even when the chemotactic bacteria are self-assembled to the micro structure, when the target material is not provided, the behavior of the micro robot of example 2 is random behavior.

### <Experimental Example 3> Analysis of Speed of Micro Robot According to Number of Self-assembled Bacteria

To identify a speed of the liquid crystal-based multifunctional micro robot according to an embodiment of the present disclosure, the behavior of the micro robot manufactured through example 2 and comparative example 2 (the first adherent micro particle/micro structure complex to which the bacteria are self-assembled) was photographed in real time using the optical microscope, the corresponding image is pre-processed, and thus a speed field of the micro robot according to the number of bacteria self-assembled to the micro structure was acquired. Here, the number of bacteria self-assembled to the micro structure satisfies a range of one to ten.

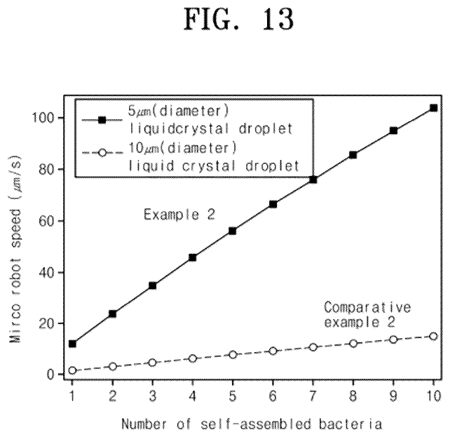

In analysis of the speed, a speed graph of the micro robot according to the number of self-assembled bacteria in a state in which the target material is provided was acquired and then illustrated in FIG. 13.

FIG. 13 is a view illustrating an experimental result for identifying a speed according to the number of self-assembled bacteria in the liquid crystal-based multifunctional micro robot according to an embodiment of the present disclosure.

Referring to FIG. 13, it was identified that the speed of the micro robot of example 2 and comparative example 2 linearly increases as the number of self-assembled bacteria increases.

In addition, it was identified that, when the micro robot is formed by adjusting the size of the micro structure, as the size of the formed micro structure decreases, the speed of the micro robot according to the number of self-assembled bacteria motors is further improved.

Accordingly, the liquid-crystal-based multifunctional micro robot according to an embodiment of the present disclosure may detect a lesion and release a treatment drug using self-power of the bacteria in a living body, thereby enabling accurate targeting by the chemotaxis and selective release of the drug.

A liquid crystal-based multifunctional micro robot according to an embodiment of the present disclosure may improve reliability and functionality in performance and functions required for a non-electronic device-based micro robot and improve self-position recognition, self-detection technology, self-movement technology, and self-drug release characteristics in a non-structural environment.

The above detailed description exemplifies the present disclosure.

Furthermore, the above-mentioned contents describe an embodiment of the present disclosure, and the present disclosure may be used in various other combinations, changes, and environments. That is, the present disclosure may be modified and corrected without departing from the scope of the present disclosure that is disclosed in the specification, the equivalent scope to the written disclosures, and/or the technical or knowledge range of those skilled in the art. An embodiment describes the best state for implementing the technical spirit of the present disclosure, and various changes required in the detailed application fields and purposes of the present disclosure may be made. Thus, the above detailed description of the present disclosure is not intended to restrict the present disclosure in an embodiment. Furthermore, it should be construed that the appended claims include an embodiment.

What is claimed is:

1. A liquid crystal-based multifunctional micro robot comprising:
a micro structure including at least one topological defect;
an adherent micro particle that is bound to the topological defect and induces self-assembly of a target including a bacterium and a target material based on an antigen-antibody reaction, a physical binding reaction, or a chemical binding reaction; and
the bacterium that is attached to the adherent micro particle and provides self-power so that the micro structure approaches the target material.

2. The liquid crystal-based multifunctional micro robot of claim 1, wherein the micro structure has a fluid mold core structure made of liquid crystal molecules and includes a carrier stored inside the micro structure.

3. The liquid crystal-based multifunctional micro robot of claim 2, wherein the micro structure has a chain core structure including a plurality of mesogenic polymer chains having a ring shape.

4. The liquid crystal-based multifunctional micro robot of claim 3, wherein the adherent micro particle is self-assembled to the at least one topological defect disposed on a preset surface area of the micro structure.

5. The liquid crystal-based multifunctional micro robot of claim 4, wherein the preset surface area is at least one pole area selected from one side hemisphere pole area and the other side hemisphere pole area of the micro structure.

6. The liquid crystal-based multifunctional micro robot of claim 5, wherein the adherent micro particle is a micro particle coated with any one material selected from an antibody specifically bound to surface protein of the target, an aptamer having the same properties as the antibody, an organic molecule specifically bound to the target, and an adsorption structure configured to adsorb the target.

7. The liquid crystal-based multifunctional micro robot of claim 6, wherein the adherent micro particle includes a first adherent micro particle that is bound to the topological defect disposed on the one side hemisphere pole area of the micro structure and provides a first attachment point to which the bacterium is attached, and a second adherent micro particle that is bound to the topological defect disposed on the other side hemisphere pole area of the micro structure and provides a second attachment point to which the target material is attached.

8. The liquid crystal-based multifunctional micro robot of claim 7, wherein at least one micro particle selected from the first adherent micro particle and the second adherent micro particle further includes a magnetic body.

9. The liquid crystal-based multifunctional micro robot of claim 8, wherein the bacterium is self-assembled to a distal end of the adherent micro particle based on the antigen-antibody reaction and moves the micro structure to an area in which the target material is positioned, through chemotaxis behavior that is a response to a stimulus due to a chemical material concentration difference.

10. The liquid crystal-based multifunctional micro robot of claim 9, wherein the bacterium moves the micro structure to the area in which the target material is positioned, through aerotaxis behavior that is a response to a stimulus due to an oxygen concentration difference and a response to a stimulus due to an oxygen saturation difference.

11. The liquid crystal-based multifunctional micro robot of claim 10, wherein the bacterium moves the micro structure to the area in which the target material is positioned, through thermotaxis behavior that is a response to a stimulus due to a temperature gradient.

12. The liquid crystal-based multifunctional micro robot of claim 11, wherein the bacterium moves the micro structure to the area in which the target material is positioned, through rheotaxis behavior that is a response to a stimulus due to a flow rate gradient.

13. The liquid crystal-based multifunctional micro robot of claim 12, wherein the bacterium moves the micro structure to the area in which the target material is positioned, through magnetotaxis behavior that is a response to a stimulus due to a magnetic field.
